(19) United States
(12) Patent Application Publication
(10) Pub. No.: US 2011/0196265 A1
(43) Pub. Date: Aug. 11, 2011

(54) FREQUENCY MODULATED ULTRASONIC THERAPEUTIC APPARATUS FOR BONE FRACTURE AND METHOD FOR GENERATING FREQUENCY MODULATED ULTRASONIC WAVE

(75) Inventors: HONG-SHONG CHANG, CHUNG LI 32023 (TW); KUAN-JUNG LI, CHUNG LI 32023 (TW); CHIUNG-CHENG CHUANG, CHUNG LI 3203 (TW)

(73) Assignee: CHUNG YUAN CHRISTIAN UNIVERSITY, CHUNG LI (TW)

(21) Appl. No.: 12/700,835

(22) Filed: Feb. 5, 2010

(51) Int. Cl. A61N 7/00

(52) U.S. Cl. 601/2

(57) ABSTRACT

A frequency modulated ultrasonic therapeutic apparatus for bone fractures and a method for generating frequency modulated ultrasonic waves are revealed. The frequency modulated ultrasonic therapeutic apparatus includes a control interface, a control unit, a waveform generator circuit and a transducer. The control interface is used to set a plurality of parameters. The control unit coupled to the control interface generates a control signal according to those parameters. The waveform generator circuit is coupled to the control unit and is generating a waveform signal according to the control signal. The transducer converts the waveform signal and produces an ultrasonic wave. The present invention provides different parameters for modulating a frequency of ultrasonic waves. Ultrasonic waves with different frequencies are used to treat bone fractures during the bone healing process of living animals. Thus the fracture healing is speeded up and the recovery time is reduced.

### BACKGROUND OF THE INVENTION

1. Fields of the Invention

The present invention relates to an ultrasonic wave, especially to a frequency modulated ultrasonic therapeutic apparatus for bone fractures and a method for generating frequency modulated ultrasonic waves.

2. Descriptions of Related Art

Generally, bones grew at early stage in human bodies are soft, flexible and plastic. Along with increasing age, the cartilage component is decreasing while the bond component is increasing so that the elder people have a higher frequency of bone fracture. Bone fractures are most often caused by falling, traffic accidents, sports, or natural disasters. When bone fracture occurs, the wound is very painful, and the wound also has an unnatural bending and obvious swelling.

Bone fracture is a common injury. During the rest period, the cost includes not only medical expenses but also income loss. Some patients sustain serious fractures may never go back to work and this lead to social problems such as unemployment, increased family burden, family financial difficulty, etc. Therapies for bone fracture require a rest period no matter traditional gypsum for fixation or operation is used. For patients and their families unable to rest for a certain period or unable to afford the long-term medical cost, if the fracture healing process can be accelerated, the reset period is shortened and the medical expenses are reduced. Thus the total load and cost caused by fractures can be decreased.

After bone fractures, the time required for bone healing is about three to five months, affected by the severity of the fracture and the patient's status. A delayed union occurs when a bone does not heal within six months after a break. When the broken bone fails to heal within nine months, it is called a nonunion. Besides the patient's status, bone healing time is also influenced by other factors such as patient's age, a time period from the fracture occurring to the treatment, fracture site, severity, blood supply, smoking, obesity, heavy drinking, nutrition status, infection, kidney disease, drug abuse and so on. According to statistics, about 10 to 20 percent of the bone fracture patients have problems of delayed union or nonunion. They need to receive the operation again or bone grafting. Thus how to shorten the healing time for reducing medical expenses and cost becomes an important issue for these patients.

The method available now for promoting bone healing stimulation by ultrasonic waves so as to shorten recovery time. However, most of ultrasonic apparatuses for accelerating bone healing use ultrasonic waves having fixed frequency to activate cells. During the bone healing process, cells are treated by the ultrasonic waves having fixed frequency. The frequency of the ultrasonic waves is not modulated within different stages of the bone healing, Although the bone healing is speed up and the healing time is shortened by the treatment compared with conventional natural process of bone healing, the treatment provides limited effects. This is due to that cells and tissues involved in different stages of the bone healing are different.

Thus there is a need to provide a novel frequency modulated ultrasonic therapeutic apparatus for bone fractures and a method for generating frequency modulated ultrasonic waves not only overcomes above shortcomings but also enhances the fracture healing and shortens the recovery of the bone fracture healing,

### SUMMARY OF THE INVENTION

Therefore it is a primary object of the present invention to provide a frequency modulated ultrasonic therapeutic apparatus for bone fractures and a method for generating frequency modulated ultrasonic waves that modulate frequency of ultrasonic waves during, the treatment of fracture healing and stimulate cells with ultrasonic waves having different frequencies for accelerating fracture healing and reducing healing time. Thus the waste of individual and social cost is reduced and patient's life quality is improved.

It is another object of the present invention to apply frequency modulated ultrasonic waves to a fracture site of living animals so as to promote cavitation and further enhance therapeutic effect of frequency modulated ultrasonic waves on bone factures,

In order to achieve above objects, a frequency modulated ultrasonic therapeutic apparatus for bone fractures of the present invention includes a control interface, a control unit, a waveform generator circuit and a transducer. The control interface is used to set a plurality of parameters and the control unit generates a control signal according to those parameters. The waveform generator circuit generates a waveform signal according to the control signal. The transducer converts the waveform signal and produces an ultrasonic wave for treating fracture of living animals. The present invention provides different parameters for modulating a frequency of ultrasonic waves, Ultrasonic waves with different frequencies are used to treat bone fractures during the bone healing process of living animals. Thus the fracture healing is speeded up and the recovery time is reduced,

Moreover, cavitation is promoted by frequency modulated ultrasonic waves acting on the bone fracture site of living animals. Thus the therapeutic effect of frequency modulated ultrasonic waves on bone fractures is further enhanced,

Furthermore, frequency modulated ultrasonic therapeutic apparatus for bone fractures healing of the present invention further includes a coupling circuit that is coupled between the waveform generator circuit and the transducer and is used for coupling the waveform signal from the waveform generator circuit. Thus the waveform signal is converted into an ultrasonic wave by the transducer.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a block diagram of an embodiment of a frequency modulated ultrasonic therapeutic apparatus for bone fractures according to the present invention;

FIG. 2 is a flow chart of an embodiment of a method for generating frequency modulated ultrasonic waves used in bone fracture treatment according to the present invention;

FIG. 3 is a block diagram of another embodiment of a frequency modulated ultrasonic therapeutic apparatus for bone fractures according to the present invention;

FIG. 4 is a flow chart of another embodiment of a method for generating frequency modulated ultrasonic waves used in bone fracture treatment according to the present invention;

FIG. 5A is a bar chart showing experimental results of the present invention;

FIG. 5B is a bar chart showing experimental results of the present invention;

FIG. 6A is a bar chart showing experimental results of the present invention; and

FIG. 6B is a bar chart showing experimental results of the present invention.

### DETAILED DESCRIPTION OF THE PREFERRED EMBODIMENT

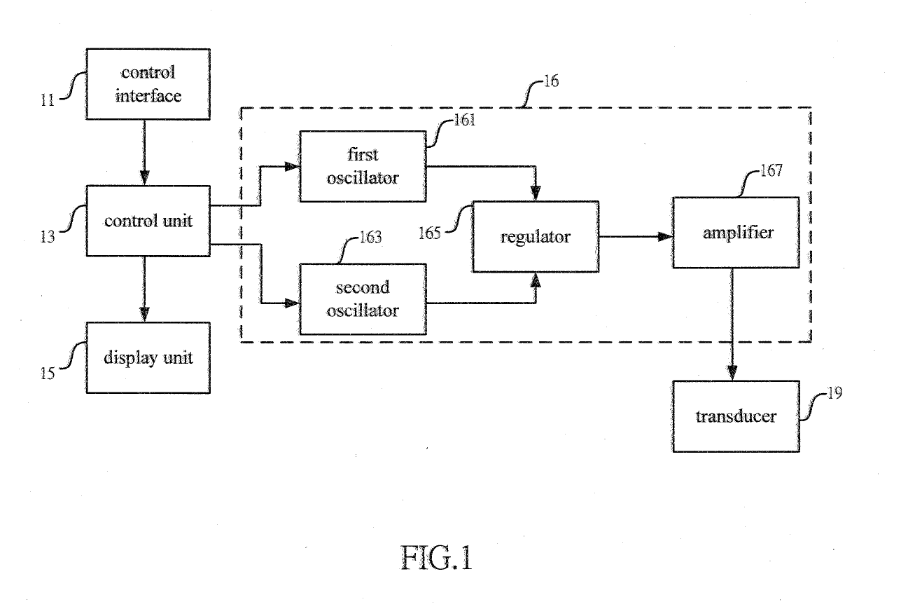

Refer to FIG. 1, a block diagram of an embodiment of a frequency modulated ultrasonic therapeutic apparatus for bone fractures according to the present invention is revealed. As shown in figure, the frequency modulated ultrasonic therapeutic apparatus for bone fractures according to the present invention includes a control interface 11, a control unit 13, a display unit 15, a waveform generator circuit 16 and a transducer 19. The control interface 11 is for users to input and set a plurality of parameters. The parameters are common parameters such as waveform signal frequency or on/off time. The control interface 11 can also display these parameters. The control unit 13 is coupled with the control interface 11, generating a control signal according to those parameters mentioned and sending the control signal to the waveform generator circuit 16. The control unit 13 also controls the display unit 15 that shows modulation related parameters for monitoring those parameters. In an embodiment of the present invention, the control interface 11 is integrated with the display unit 15. The control unit 13 of the present invention can be a beamforming control unit.

Still FIG. 1, the waveform generator circuit 16 is coupled with the control unit 13 for receiving a control signal from the control unit 13 and generating a waveform signal. The control unit 13 generates a corresponding control signal according to the parameters from the control interface 11 so that the waveform generator circuit 16 generates the waveform signal according to these parameters that defines characters of the waveform signal such as frequency and on/off time. The transducer 19 is used to convert the waveform signal for generating an ultrasonic wave. The transducer 19 is a probe of the ultrasonic therapeutic apparatus and it can be a piezoelectric transducer in a preferred embodiment.

At each stage of the fracture healing process, cells and tissues involved are different. Thus different parameters are set in the present invention at different stages of the fracture healing process. Thus according to different parameters, the control unit 13 adjusts a frequency of the waveform signal so as to modulate the frequency of the ultrasonic waves generated by the transducer 19. Thus during the bone healing, ultrasonic waves with different frequencies are used to simulate cells so as to activate cells for accelerating the bone healing and further reducing reduce the recovery time for injuries. Moreover, the present invention is applied not only to bone fracture therapy of humans, but also to bone fracture therapy of other animals. Thus the present invention is for treatment of bone fracture of all living animals.

In an embodiment of the present invention, the recovery process is divided into a plurality of recovery stages. Different control parameters are set corresponding to each recovery stage so as to module frequencies of ultrasonic waves. The modulation of frequency is from high frequency to low frequency. For example, the fracture healing process is divided into a first recovery stage, a second recovery stage, and a third recovery stage. Ultrasonic frequencies corresponding to the above three stages are 3.0 MHz, 1.5 MHz and 0.75 MHz respectively. The above embodiment is a only preferred embodiment, not intended to limit the present invention. Furthermore, by adjustment of parameters, a duty cycle of the waveform signal is modulated and the ratio of on/off time of the waveform signal is further adjusted. In an embodiment, the ratio of on/off time of the waveform signal is 1:4.

Back to FIG. 1, the waveform generator circuit 16 consists of a first oscillator 161, a second oscillator 163 and a regulator 165. The first oscillator 161 is coupled to the control unit 13 and is generating a first waveform signal according to the control signal. In an embodiment of the first waveform signal, it can be a rectangular wave signal. The first oscillator 161 modulates a duty cycle of the first waveform signal according to the control signal. The second oscillator 163 is coupled to the control unit 13 and is generating a second waveform signal according to the control signal. In an embodiment of the second waveform signal, it can be a sine wave or a pulse signal. The second oscillator 163 also modulates a frequency of the second waveform signal according to the control signal.

The control signals are generated by the control unit 13 according to parameters to control the first oscillator 161 as well as the second oscillator 163. Thus according to the parameters, the first oscillator 161 and the second oscillator 163 respectively generate a first waveform signal for modulating the duty cycle and a second waveform signal for modulating the frequency, The regulator 165 is coupled to the first oscillator 161 and the second oscillator 163 for receiving the first waveform signal from the first oscillator 161 as well as the second waveform signal from the second oscillator 163 and generating a waveform signal. The first waveform signal is used as a switch signal and the regulator 165 outputs the second waveform signal of the second oscillator 163 according to “on” time of the switch signal. The first oscillator 161 of the frequency modulated ultrasonic therapeutic apparatus for bone fractures is a multivibrator. Moreover, the waveform generator circuit 16 further includes an amplifier 167 that is coupled to the regulator 165 for amplifying waveform signals generated by the regulator 165 to be converted by the transducer 19. Thus ultrasonic waves are generated.

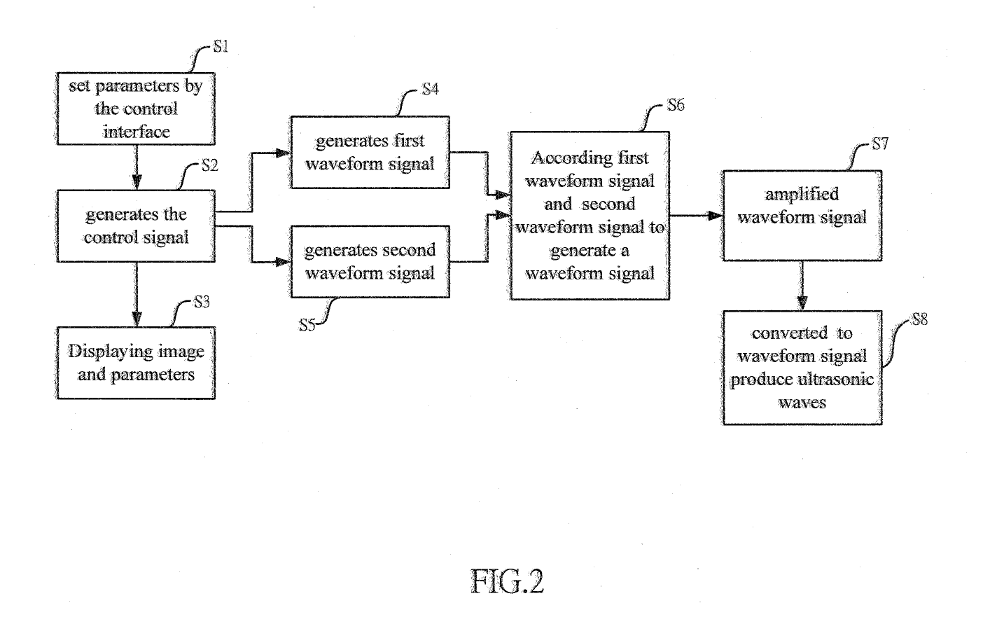

Refer to FIG. 2, a flow chart of a method for generating frequency modulated ultrasonic waves used in bone fracture treatment is revealed. As shown in figure, firstly refer to the step S1, set parameters by the control interface 11. This step provides different parameters during the bone recovery process of living animals. The parameters set are displayed on the control interface 11. Then a waveform signal is generated according to those parameters, as shown in the step S2, step S4, step S5 and step S6. The control unit 13 generates the control signal in accordance with those parameters. Later a first waveform signal that modulates the duty cycle and a second waveform signal that modulates the frequency are generated by the waveform generator circuit 16 according to the control signal. That means according the parameters, the first waveform signal for modulating the duty cycle and the second waveform signal for modulating the frequency are generated respectively. Next the regulator 165 modulates the first waveform signal and the second waveform signal to generate a waveform signal. The regulator 165 outputs the waveform signal according to the duty cycle of the first waveform signal that works as a switch signal. As shown in the step S7, after generation of the waveform signal, the waveform signal is amplified by the amplifier 167. The waveform signal generated according to those parameters is then converted by the transducer 19 to produce ultrasonic waves. Moreover, as shown in the step S3, images and modulation parameters are displayed an the display unit 15,

The present invention treats bone fractures by ultrasonic waves that are acoustic waves that generate little force acting on the fracture and stimulating bones to adapt and change its structure. At the same time, tissues absorb energy from ultrasonic waves and the frequency of the ultrasonic waves is adjusted according to time period of the injury. Thereby metabolism is reconstructed or normalized so as to speed up body fluid around for accelerating nutrition supply and waste removal. The ultrasonic waves can also regulate gene expression and increase callus formation. Moreover, ultrasonic waves increase blood flow velocity and promote growth of new blood vessels. The increasing of blood flow also enhances fracture healing. Due to different cells and tissue compositions involved during different stages of the fracture healing, parameters set are changed according to fracture healing status in the present invention. The frequency modulated ultrasonic waves are used to treat bone fractures for enhancement of fracture healing.

The therapeutic effect of ultrasonic waves on bone fractures is due to ultrasonic cavitation that affects callus formation and enhances bone healing. Thus the frequency modulated ultrasonic waves of the present invention can be used in combination with in vitro micro/nanobubbles injected. That means the frequency modulated ultrasonic waves act on the plurality of micro/nanobubbles at the fracture site of living animals so as to promote cavitation effects. Thus the therapeutic effect of frequency modulated ultrasonic waves on bone fractures is further enhanced. Furthermore, the present invention can further make some modifications on surfaces of the micro/nanobubbles for tageting of a treatment of bone fracture sites.

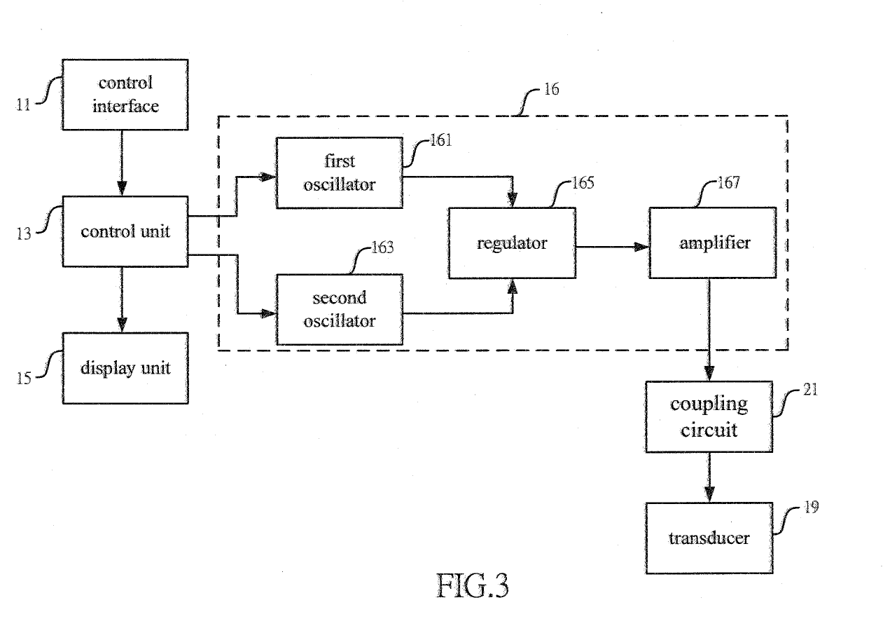

Refer to FIG. 3, a block diagram of another embodiment is disclosed. As shown in figure, the difference between this embodiment and the above one is in that: a frequency modulated ultrasonic therapeutic apparatus for bone fractures of this embodiment further includes a coupling circuit 21 that is coupled between the waveform generator circuit 16 and the transducer 19. In order to prevent that the waveform signal generated by the waveform generator circuit 16 is not compatible with the receiving transducer 19, the coupling circuit 21 is for coupling the waveform signals from the waveform generator circuit 16 and sending the coupled waveform signals to the transducer 19 for conversion.

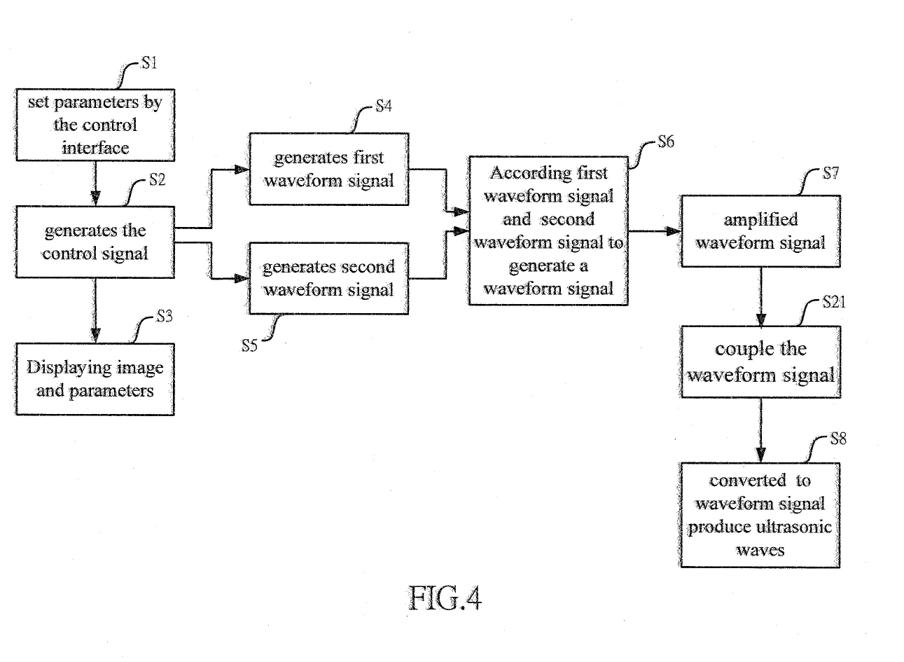

Refer to FIG. 4, a flow chart of another embodiment of a method for generating frequency modulated ultrasonic waves used in bone fracture treatment is disclosed. As shown in figure, the different between this method and the one in FIG. 2 is in that: this embodiment further includes a step S21-couple the waveform signal by the coupling circuit 21 for following conversion and production of ultrasonic waves.

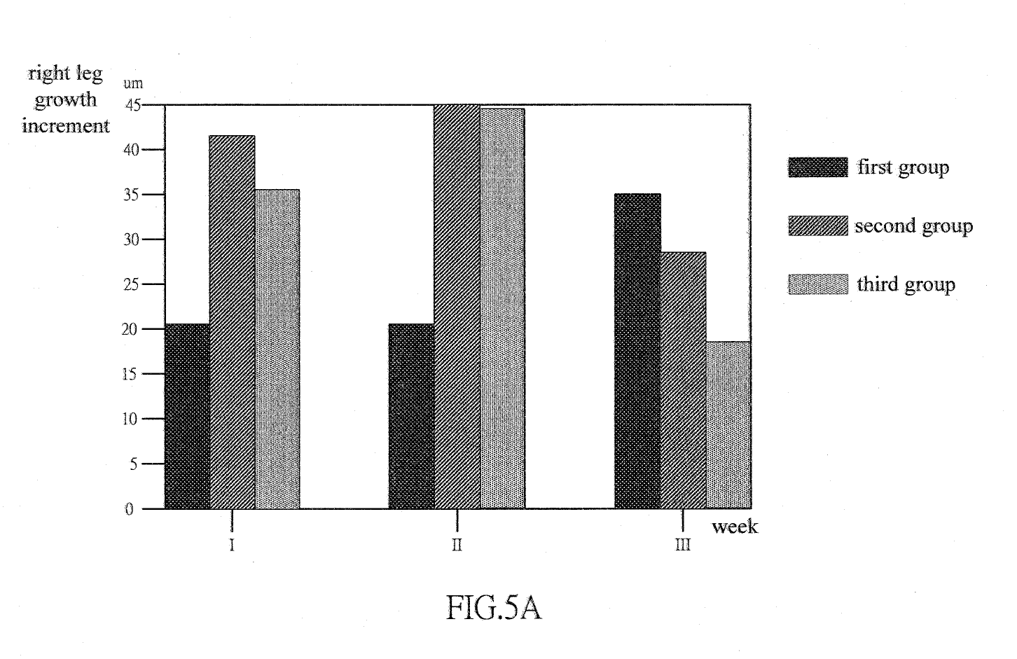

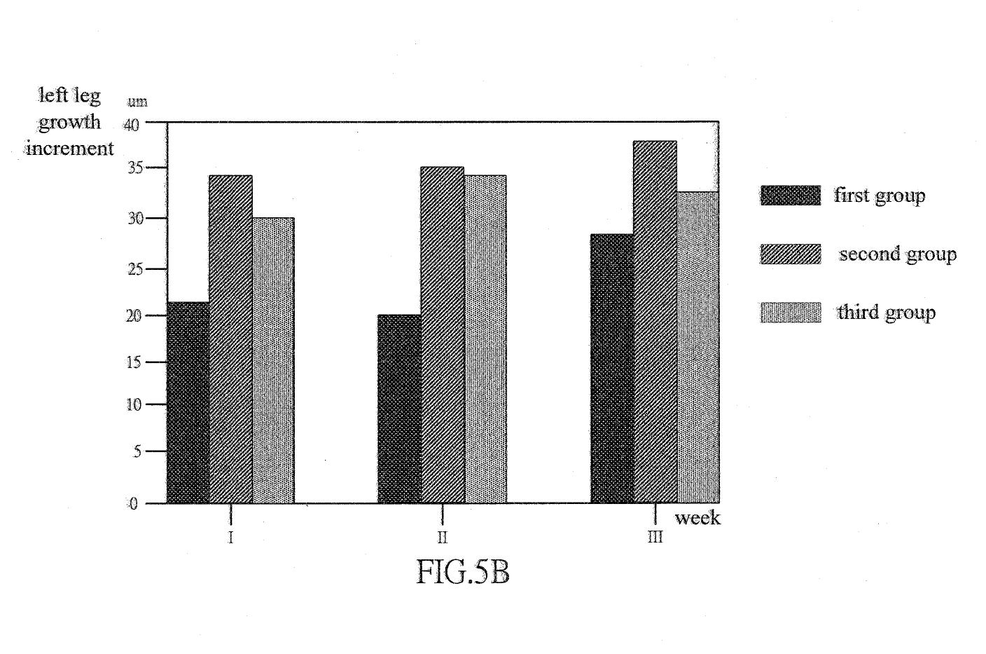

Refer to FIG. 5A and FIG. 5B, bar charts showing experimental results of the present invention with therapeutic ultrasonic waves, conventional fixed therapeutic ultrasonic waves, and no ultrasonic waves on bone fractures of rabbits are displayed. A plurality of rabbits is used as fibula fracture models in the experiment after operations. According to different treatments, the rabbits are divided into three groups. The first group is healing naturally, without any treatment. The second group is frequency modulated group. The present invention is used and various recovery stages of bone fracture are applied with ultrasonic waves having different frequencies that are decreasing gradually. The third group is a continuous wave group. The rabbits are applied with ultrasonic waves having the same frequency during the fracture healing process. During the treatment, the left leg of each rabbit is applied with the same pressure without treatment. This is for comparison. As to the right leg, it is applied with ultrasonic wave treatment. In the first group, both right and left legs are not treated.

Refer to FIG. 5A, a growth increment of right leg bones of each group within a single week is shown. It is learned from the figure that right leg bone of the third group rabbits that are treated with therapeutic ultrasonic waves having fixed frequency grows better than that of the first group rabbits without any treatment. Moreover, the growth increment of right leg bones of the second group rabbits that are treated with therapeutic ultrasonic waves having modulated frequencies is larger than that of the right leg bones of the third group rabbits. Thus during the fracture healing, the therapeutic effect of the frequency modulated ultrasonic waves on the bone fractures is better than the effect of the fixed frequency ultrasonic waves on the bone fractures. Also refer to FIG. 5B, a growth increment of left leg bones of each group rabbits is shown, As shown in figure, although the left legs of rabbits don't receive any treatment, the growth increment of left legs is also promoted while the ultrasonic waves stimulating the right legs. It is obvious that the therapeutic effect of the frequency modulated ultrasonic waves is better than that of the fixed frequency ultrasonic waves.

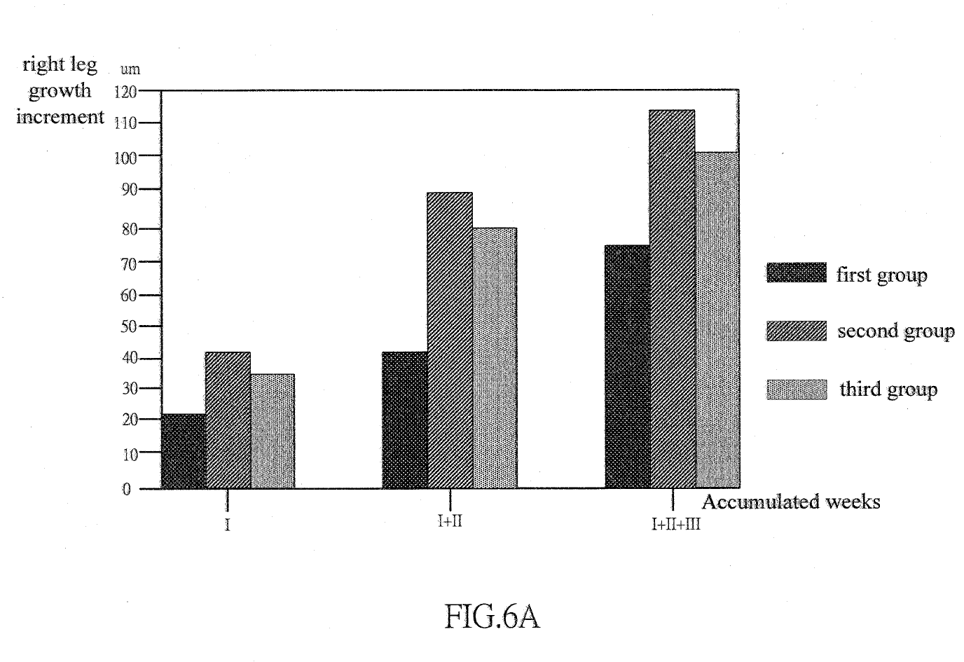

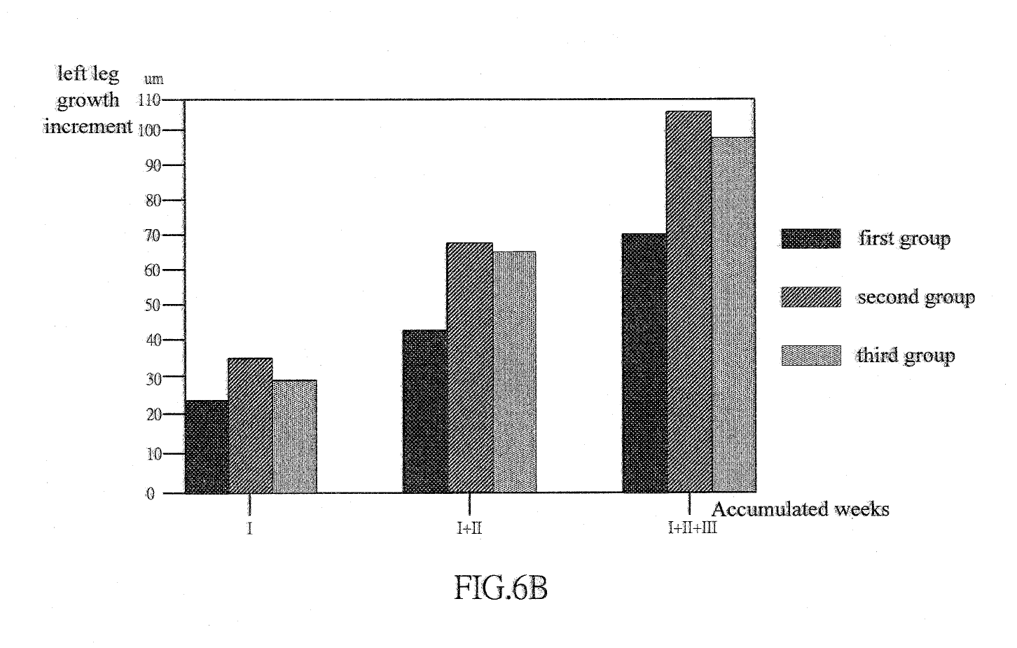

Refer to FIG. 6A and FIG. 6B, bar charts showing experimental results of the present invention with therapeutic ultrasonic waves, conventional fixed therapeutic ultrasonic waves, and no ultrasonic waves on bone fractures of rabbits are displayed. FIG. 6A shows the total growth increment of right leg bones of rabbits within three weeks. The right leg bone of the third group rabbits that are treated with fixed frequency ultrasonic grows better than that of the first group rabbits without any treatment. Furthermore, the total growth increment of the second group rabbits treated with modulated frequency ultrasonic growth is larger than that of the third group rabbits. Also refer to FIG. 6B, it shows the total growth increment of left leg bones of rabbits within three weeks. As shown in figure, although the left legs of rabbits don't get any treatment, the total growth increment of left legs is also increased while the ultrasonic waves stimulating the right legs.

In summary, the frequency modulated ultrasonic therapeutic apparatus for bone fractures of the present invention includes a control interface, a control unit, a waveform generator circuit and a transducer. The control interface is for setting a plurality of parameters and the control unit generates a control signal according to the parameters and the control signal is sent to the waveform generator circuit. A waveform signal is generated according to the control signal and is sent to the transducer for conversion and production of ultrasonic waves. The present invention mainly provides different parameters of frequency modulation of ultrasonic waves so as to use ultrasonic waves with different frequencies stimulating cells, accelerating fracture healing and further reducing healing time during the bone healing process of living animals. Moreover, the frequency modulated ultrasonic waves is applied to and acted on a plurality of micro/nanobubbles at the fracture site so as to promote cavitation and further enhance therapeutic effect of frequency modulated ultrasonic waves on bone factures.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A frequency modulated ultrasonic therapeutic apparatus for a bone fracture of a living animal comprising:
a control interface used to set a plurality of parameters and display the parameters;
a control unit coupled to the control interface and used for generating a control signal according to the parameters;
a waveform generator circuit coupled to the control unit and generating a waveform signal according to the control signal; and
a transducer that converts the waveform signal to produce an ultrasonic wave;
wherein the control interface set different parameters during a fracture healing of the living animal and the control unit modulates a frequency of the waveform signal according to the different parameters so as to module a frequency of the ultrasonic wave; thus the bone fractures is treated by the ultrasonic wave with different frequencies during the fracture healing of the living animal.

2. The device as claimed in claim 1, wherein the fracture healing includes a plurality of recovery stages and different parameters are set corresponding to the recovery stages for modulation of the frequency of the ultrasonic wave; the frequencies of the ultrasonic wave corresponding to the recovery stages are from high frequency to low frequency so as to treat the bone fracture of the living animal.

3. The device as claimed in claim 2, wherein the plurality of recovery stages includes a first recovery stage, a second recovery stage, and a third recovery stage while the frequency of the ultrasonic wave corresponding to the first recovery stage, the second recovery stage, and the third recovery stage is 3.0 MHz, 1.5 MHz and 0.75 MHz respectively.

4. The device as claimed in claim 1, wherein the frequency modulated ultrasonic therapeutic apparatus for a fracture further includes:
a display unit that shows an image and the parameters,

5. The device as claimed in claim 4, wherein the control interface is integrated with the display unit.

6. The device as claimed in claim 1, wherein the waveform generator circuit comprising:
a first oscillator coupled to the control unit and generating a first waveform signal according to the control signal;
a second oscillator coupled to the control unit and generating a second waveform signal according to the control signal; and
a regulator coupled to the first oscillator and the second oscillator for receiving the first waveform signal as well as the second waveform signal and generating the waveform signal.

7. The device as claimed in claim 6, wherein the first oscillator modulates a duty cycle of the first waveform signal and the second oscillator modulates a frequency of the second waveform signal.

8. The device as claimed in claim 6, wherein the first waveform signal is a rectangular wave signal and the second waveform signal is a sine wave or a pulse signal.

9. The device as claimed in claim 6, wherein the waveform generator circuit further comprising:
an amplifier that is coupled to the regulator and amplifying the waveform signal.

10. The device as claimed in claim 6, wherein the first oscillator is a multivibrator.

11. The device as claimed in claim 1, wherein the control unit is a beamforming control unit.

12. The device as claimed in claim 1, wherein the transducer is a probe.

13. The device as claimed in claim 1, wherein the transducer is a piezoelectric transducer.

14. The device as claimed in claim 1, wherein the frequency modulated ultrasonic therapeutic apparatus for bone fractures further includes: a coupling circuit that is coupled between the waveform generator circuit and the transducer and is used for coupling the waveform signal generated by the waveform generator circuit

15. The device as claimed in claim 1, wherein a site of the bone fracture of the living animal includes a plurality of micro/nanobubbles and the ultrasonic wave acts on the micro/nanobubbles.

16. A method for generating frequency modulated ultrasonic waves that are applied to treat a bone fracture of a living animal comprising the steps of:
providing a plurality of parameters;
generating a waveform signal according to the parameters; and
converting the waveform signal to an ultrasonic wave;
wherein different parameters are provided during a fracture healing of the living animal and a frequency of the waveform signal is modulated according to the different parameters so as to module a frequency of the ultrasonic wave; thus the bone fractures is treated by the ultrasonic wave with different frequencies during the fracture healing of the living animal.

17. The method as claimed in claim 16, wherein the fracture healing includes a plurality of recovery stages and different parameters are provided corresponding to the recovery stages for modulation of the frequency of the ultrasonic wave; the frequencies of the ultrasonic wave corresponding to the recovery stages are from high frequency to low frequency so as to treat the bone fracture of the living animal.

18. The method as claimed in claim 16, wherein the plurality of recovery stages includes a first recovery stage, a second recovery stage, and a third recovery stage while the frequency of the ultrasonic wave corresponding to the first recovery stage, the second recovery stage, and the third recovery stage is 3.0 MHz, 1.5 MHz and 0.75 MHz respectively.

19. The method as claimed in claim 16, wherein the method further comprising a step of displaying the parameters.

20. The method as claimed in claim 16, wherein the step of generating a waveform signal according to the parameters further includes a step of:
generating a control signal according to the parameters so as to produce the waveform signal according to the control signal.

21. The method as claimed in claim 16, wherein the step of generating a waveform signal according to the parameters further includes steps of:
generating a first waveform signal according to the parameters,
generating a second waveform signal according to the parameters, and
generating the waveform signal according to the first waveform signal and the second waveform signal,

22. The method as claimed in claim 21, wherein. in the steps of generating a first waveform signal as well as a second waveform signal according to the parameters, a duty cycle of the first waveform signal and a frequency of the second waveform signal are modulated according to the parameters.

23. The method as claimed in claim 21, wherein the first waveform signal is a rectangular wave signal and the second waveform signal is a sine wave or a pulse signal.

24. The method as claimed in claim 21, wherein the method further comprising a step of: amplifying the waveform signal.

25. The method as claimed in claim 16, wherein the method further comprising a step of: coupling the waveform signal for converting the waveform signal to generate the ultrasonic wave.

26. The method as claimed in claim 16, wherein a site of the bone fracture of the living animal includes a plurality of micro/nanobubbles and the ultrasonic wave acts on the micro/nanobubbles.
